(19) United States
(12) Patent Application Publication
(10) Pub. No.: US 2007/0137726 A1
(43) Pub. Date: Jun. 21, 2007

(54) BEVERAGE DISPENSER

(75) Inventor: Chan Man Yan, Hunghorn (HK)

Correspondence Address: HOGAN & HARTSON L.L.P., 1999 AVENUE OF THE STARS, SUITE 1400, LOS ANGELES, CA, 90067, US

(73) Assignee: C. Art Ltd.

(21) Appl. No.: 11/534,316

(22) Filed: Sep. 22, 2006

(30) Foreign Application Priority Data
Oct. 24, 2005 (HK) 05109460.7
Oct. 24, 2005 (HK) 05109461.6
Apr. 6, 2006 (HK) 06104215.5
Apr. 7, 2006 (HK) 06104292.1
Apr. 27, 2006 (HK) 06105062.6
Apr. 6, 2006 (HK) 06104216.4

(51) Int. Cl. B65B 1/04; A61L 2/00; B65B 3/04; B67D 1/08; B67D 1/07

(52) U.S. Cl. 141/85; 141/90; 222/148; 422/24; 141/98; 222/192

(57) ABSTRACT

A beverage dispenser includes an outlet for dispensing beverages and a tray for supporting a container underneath the outlet. An ultraviolet light source is directed at the outlet for disinfecting the outlet. A light source illuminates a path of a beverage between the outlet and the tray. Exterior lights are mounted on an exterior of the dispenser. A cradle secures an appliance module to the dispenser and includes an interface for communication of data or power between the appliance module and the dispenser. A bottle storage cabinet may be formed underneath the dispensing bay for storing a bottle. The storage cabinet includes a door for opening and closing the cabinet, a movable trolley for supporting and moving the bottles and a hinged ramp to facilitate entry and exit of the trolley into and out of the cabinet. Alternatively, for counter top applications, a built-in mover is formed underneath the dispenser to facilitate easy movement of the dispenser.

### RELATED APPLICATIONS

This application claims the benefit of priority under 35 USC 119 of Hong Kong patent application no. 05109460.7, filed on Oct. 24, 2005, P.R.C patent application no. HK:05351 Hong Kong patent application no. 05109461.6, filed on Oct. 24, 2005, P.R.C patent application no. HK:05352 Hong Kong patent application no. 06104215.5, filed on Apr. 6, 2006, P.R.C patent application no. HK: (t.b.a) Hong Kong patent application no. 06104292.1, filed on Apr. 6, 2006, P.R.C patent application no. HK: 06153 Hong Kong patent application no. 06105062.6, filed on Apr. 7, 2006, P.R.C patent application no. HK: 06180 Hong Kong patent application no. 06104216.4 filed on Apr. 6, 2006, P.R.C patent application no. HK: 06152, which applications are hereby incorporated by reference in their entireties.

### BACKGROUND OF THE INVENTION

1. Field of the Invention

The present invention relates to a beverage dispenser with improved utilitarian, entertainment and safety features.

2. Description of Related Art

Beverage dispensers are often used in homes, workplaces, food outlets, clubs, etc. as a source of chilled and/or hot water or other liquids for drinking, making beverages such as tea, coffee, etc., and for making instant foods such as noodles, soup, etc. In a typical beverage dispenser, a bottle is mounted above a housing and enclosure, and faucet outlets in communication with the enclosure dispense beverages into containers underneath the outlets.

Known beverage dispensers are suitable for providing basic manual dispensation of liquids, but are lacking in other utilitarian, entertainment and safety features.

### SUMMARY OF THE INVENTION

The present invention provides an improved beverage dispenser with entertainment, utilitarian and safety features beyond mere dispensation of beverages.

One embodiment of the invention is a beverage dispenser comprising a faucet outlet for dispensing beverages and an ultraviolet light source directed at the outlet for disinfecting the outlet. According to this embodiment, the faucet outlet is advantageously sterilized and any germs, bacteria and disease are eliminated.

Another embodiment of the invention is a beverage dispenser comprising an outlet for dispensing beverages, a tray for supporting a container underneath the outlet, and a light source for illuminating a path of a beverage between the outlet and the tray. According to this embodiment, liquid exiting the outlet has a visually striking and pleasing effect.

Another embodiment of the invention is a water dispenser comprising a dispensing bay including faucet outlets mounted above a container support tray, and a water bottle storage cabinet underneath the dispensing bay for storing a water bottle. The storage cabinet includes a door for opening and closing the cabinet, a movable trolley for supporting and moving the water bottle, and a hinged ramp to facilitate entry and exit of the trolley into and out of the cabinet. According to this embodiment, the burden on a user in moving heavy water bottles is eased.

Another embodiment of the invention is a beverage dispenser comprising a dispensing bay for dispensing beverages, a cradle for securing an appliance module to the dispenser, and an interface for communication of data or power between the appliance module and the dispenser. The appliance module may be selected, for example, from a group of interchangeable appliance modules such as a CD player, a soothing sounds player, an alarm clock and a radio. According to this embodiment, a user is able to provide the water dispenser with entertainment and/or utility functionality of his/her choosing.

Another embodiment of the invention is a beverage dispenser comprising a dispensing bay for dispensing beverages and a built-in mover underneath the dispensing bay to facilitate easy movement of the dispenser. This embodiment is particularly applicable to smaller, more mobile beverage dispensers intended for deployment on counter or table tops.

Another embodiment of the invention advantageously combines these features to provide a beverage dispenser with synergistic utilitarian, entertainment and safety features. An ultraviolet light source is directed at the outlet for disinfecting the outlet. A light source illuminates a path of a beverage between the outlet and the tray. Exterior lights are mounted on an exterior of the dispenser. A recessed cradle for securing an appliance module to the dispenser, and an interface for communication of data or power between the appliance module and the dispenser are also provided. In a stand-alone implementation, a bottle storage cabinet with a removable trolley for supporting a water bottle is formed in a lower portion of the dispenser. In a counter-top implementation, a built-in mover is formed at the bottle dispenser to facilitate easy movement of the dispenser across a counter-top.

Other features and advantages of the invention will be apparent from the following detailed description, taken in conjunction with the accompanying drawings which illustrate, by way of example, various features of embodiments of the invention.

### BRIEF DESCRIPTION OF THE DRAWINGS

FIG. 1 is a perspective view of a beverage dispenser according to a first embodiment of the invention.

FIG. 2 is a perspective view of the beverage dispenser of FIG. 1 with a water bottle mounted for dispensing water.

FIGS. 3A-C are perspective views of the beverage dispenser of FIG. 1, depicting the bottle storage cabinet and movable trolley.

FIGS. 4A-D are front views of interchangeable cassettes for the beverage dispenser of the present invention.

FIG. 5 is a perspective view of a beverage dispenser according to a second embodiment of the invention.

FIG. 6 is an exploded perspective view of the built-in mover at the bottom of the beverage dispenser of the second embodiment.

### DETAILED DESCRIPTION OF THE INVENTION

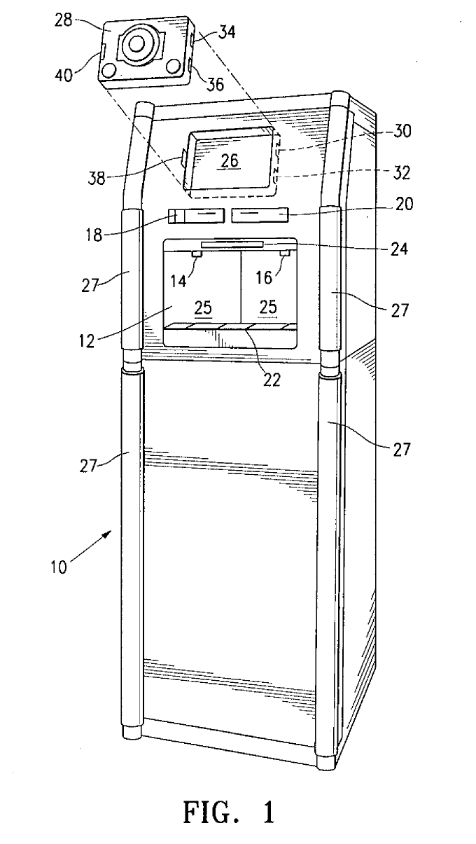

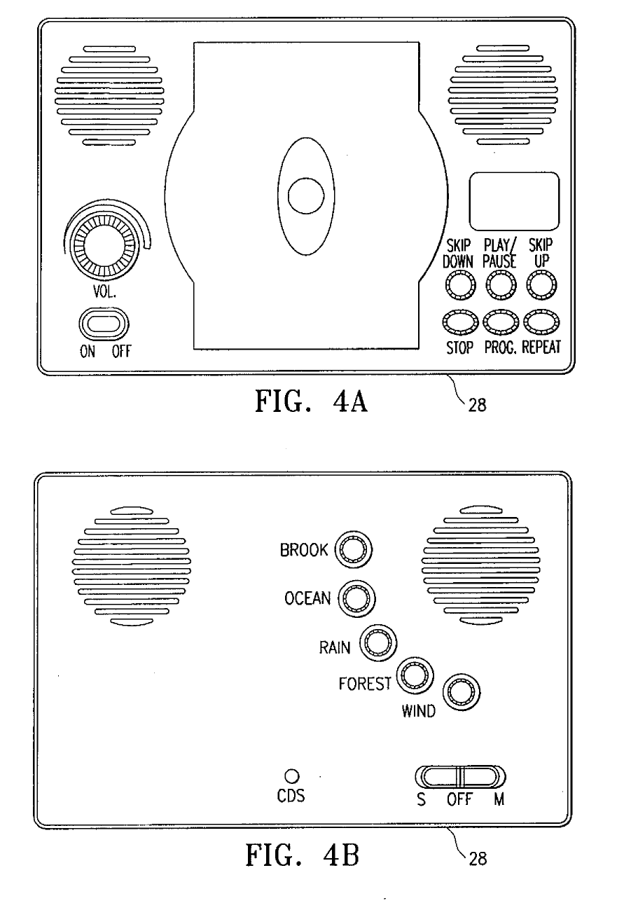

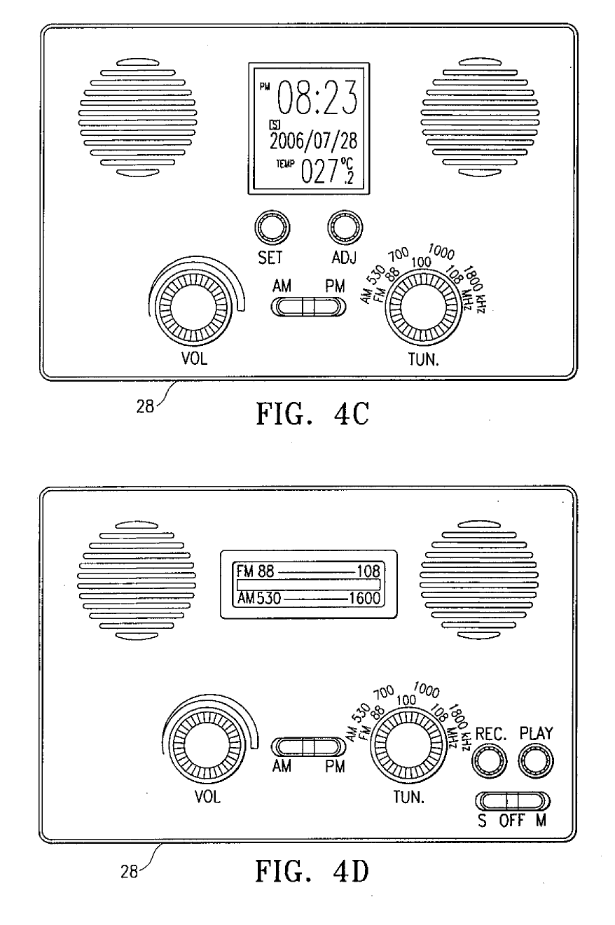

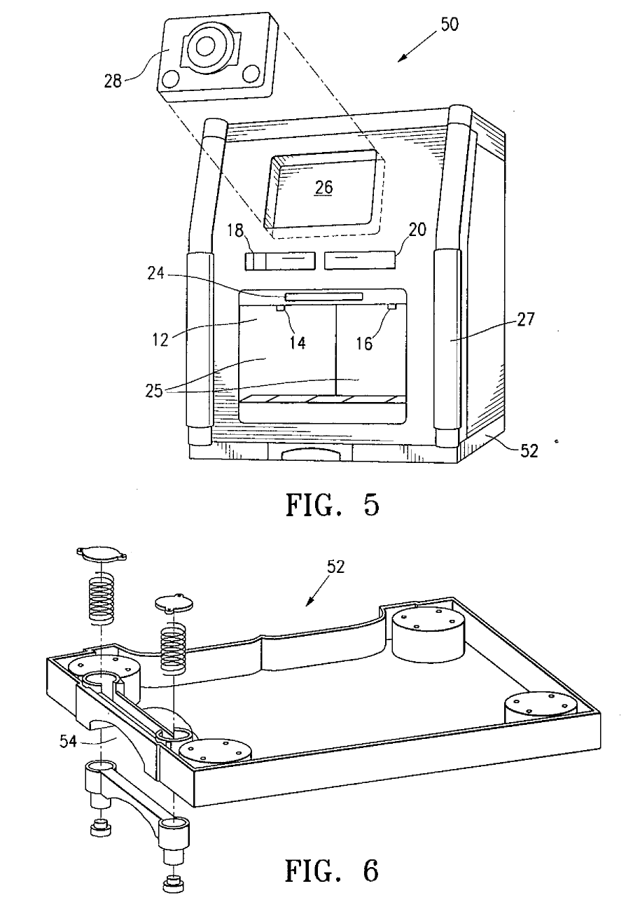

FIGS. 1-3 depict a beverage dispenser according to a first embodiment of the invention, which is typically a “stand alone” beverage dispenser that is positioned at a semi-permanent location of a home, office or other venue and not often moved. FIGS. 5-6 depict a second embodiment of the invention, which is a smaller, more mobile beverage dispenser that may be placed on a counter or table top, for example, and is easily movable. FIGS. 4A-D depict interchangeable modules or cassettes with various media functions (CD player, soothing sounds, alarm clock, radio, etc.) that may be mounted in a corresponding cradle or receptacle of the beverage dispensers of either the first or second embodiments.

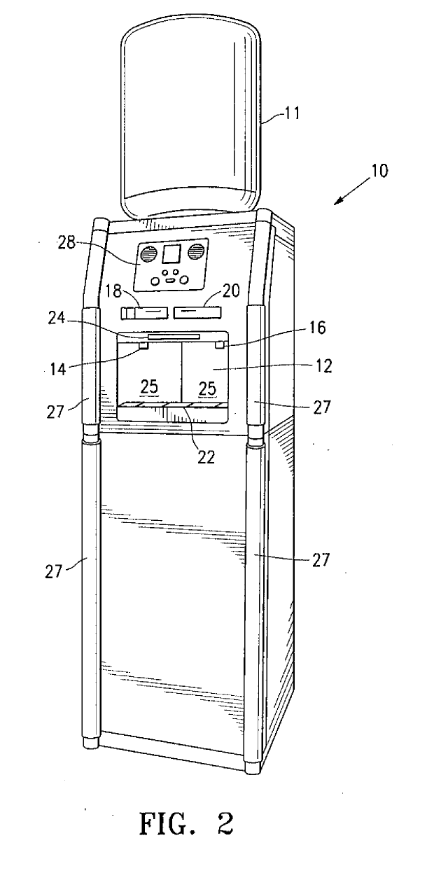

FIGS. 1-3 depict a beverage dispenser 10 according to a first embodiment of the invention. A recessed dispensing bay 12 is provided in the front of dispenser 10 and includes faucet outlets 14 and 16 for dispensing beverages. Beverage dispenser 10 may have internal refrigeration and heating means. Thus, for example, outlet 14 may dispense hot water and outlet 16 may dispense chilled water. As shown in FIG. 2, a water bottle 11 is typically mounted above dispenser 10 and dispenses water into enclosures within dispenser 10 which, in turn, are in communication with faucet outlets 14 and 16. Alternatively, outlet 14 may dispense one type of beverage and outlet 16 may dispense another type of beverage.

Faucet outlets 14 and 16 are operated by buttons 18 and 20 above dispensing bay 12. Buttons 18 and 20 may be configured to effect dispensation of liquid from outlets 14 and 16 so long as buttons 18 and 20 are depressed. Alternatively, buttons 18 and 20 may be configured to effect dispensation of a predetermined amount of liquid with a single press. Tray 22 is situated at the bottom of bay 12 underneath faucets 14 and 16 and is intended to support cups or other containers to capture beverages dispensed from faucet outlets 14 and 16.

An ultraviolet (UV) light tube 24 is positioned in dispenser 10 in close proximity to faucet outlets 14 and 16 for purposes of sterilizing faucet outlets 14 and 16. One or several UV light tubes 24 may be provided to direct UV light towards faucet outlets 14 and 16. After a user has finished using faucet outlets 14 and 16, UV light tube 24 is automatically illuminated to disinfect faucet outlets 14 and 16. Tray 22 and other areas of dispensing bay 12 may be sterilized as well. In this manner, any germs left on faucet outlets 14 and 16 by the user are killed, and accumulations of bacteria and disease are eliminated.

Automatic means (not shown) are provided for illumination of UV light tube 24 after a user has finished using faucets 14 and 16. For example, UV light tube 24 may be automatically illuminated when buttons 18 and/or 20 are released. Or, where buttons 18 and 20 are configured to effect dispensation of a predetermined amount of liquid with a single press, UV light tube 24 may be automatically illuminated after the predetermined amount of liquid has been dispensed. In another embodiment, a sensor may be provided at an appropriate location within bay 12 such as, for example, underneath tray 22. When a cup is removed from tray 22, UV light tube 24 is automatically illuminated.

UV light tube 24 is operated by a timer circuit or microcontroller (not shown) to stay illuminated for a fixed interval after operation of the faucet outlets has ceased, or a cup has been removed from the tray. The interval should be sufficient to thoroughly disinfect the faucet outlets and/or the tray and dispensing bay. In addition, light tube 24 may be triggered to illuminate after a predetermined period of nonuse of dispenser 10. A user-activated switch may also be provided to allow a user to manually activate UV light tube 24.

In addition to UV light tube 24, light sources 25 may be provided to illuminate liquids as they are dispensed from faucet outlets 14 and 16. Light sources 25 are preferably directed downwardly to illuminate the water path between outlets 14 and 16 and tray 22 to provide a visually striking and pleasing effect. In one embodiment, mood light 25 emits a blue light, so that running water has an illuminated blue appearance. In another embodiment, a red light source is provided to illuminate the path of hot water exiting one faucet, while a blue light source is provided to illuminate the path of cold water exiting the other faucet.

Additional light tubes 27 may be configured on the exterior of dispenser 10 to provide additional effects. As depicted, light tubes 27 may be positioned on the front sides of dispenser 10. The display and effects provided by light tubes 27 may vary depending on the intended venue and use of dispenser 10. For example, where dispenser 10 is to be deployed in a venue such as a nightclub or disco, lights 27 may be fluorescent, colorful and may blink or have a timed pattern. Alternatively, where dispenser 10 is to be deployed in a venue such as a workplace, lights 27 may be a simple white light.

A recessed cradle or interchangeable cassette box 26 is also provided in the front of dispenser 10. As depicted, cradle 26 is above dispensing bay 12. However, cradle 26 could be located at alternative positions as appropriate in dispenser 10. Cradle 26 is recessed within dispenser 10 and is shaped and sized to receive therein an appliance module or cassette 28. Electrical interface sockets 30 and 32 are provided within cradle 26 for electrical connection with mating interface plugs 34 and 36 formed on module 28. The interface plugs and sockets are formed with appropriate pins and receptacles for communication of data, audio, video and/or power signals. A locking tab 38 or other appropriate means is formed in cradle 26, an may engage a corresponding recess or indent in module 28, to retain module 28 within cradle 26.

As depicted in FIGS. 4A-D, appliance module 28 may be, for example, a CD player (FIG. 4A); a soothing sounds box (FIG. 4B); an alarm clock with date and/or temperature (FIG. 4C) or a radio (FIG. 4D). Preferably, a plurality of interchangeable appliance modules or cassettes 28 are provided, and may have various combinations of the above functions, as well as other functions such as DVD players, audio players, MP3 players, message centers or other communication devices, television or video screens for displaying advertisements, videos or other content, lights, etc. These are just a few of the possibilities.

As shown in FIGS. 4A-D, each appliance module 28 has user controls and buttons, audio speakers and a visual display where appropriate. In addition, or alternatively, dispenser 10 may have inbuilt speakers, displays or user interfaces for use in coordination with module 28. Module 28 may be self-powered by internal batteries or, alternatively, include a power input socket to receive power from the power source of dispenser 10. Appliance modules or cassettes 28 may be sold separately from beverage dispenser 10 and purchased according to the desire of the user of dispenser 10.

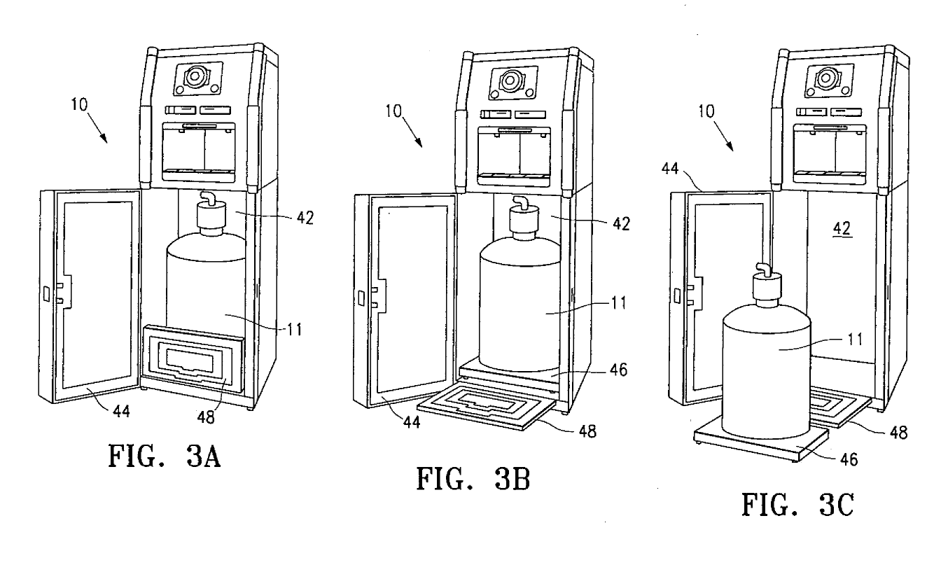

As shown in FIGS. 3A-C, a storage cabinet 42 is formed in the lower portion of dispenser 10 for storing a bottle 11 therein. A hinged door 44 is provided to enclose and provide access to cabinet 42. A movable trolley 46 is provided within cabinet 42. Bottle 11 is placed on movable trolley 46 and thus may be easily moved into and out of cabinet 42. This advantageously eases the burden on the consumer since full water bottles are quite heavy. A hinged ramp 48 is provided at the bottom of cabinet 42 to facilitate entry and exit of trolley 46 and bottle 11 into and out of cabinet 42.

FIGS. 5 and 6 depict a beverage dispenser 50 according to a second embodiment of the invention. Relative to beverage dispenser 10, dispenser 50 is a smaller and more mobile dispenser and is intended for placement on a surface such as a counter or table top.

Despite its smaller size, dispenser 50 shares many of the same features as dispenser 10 and the same reference characters are used to designate those features. For example, dispenser 50 includes a dispensing bay 12 with a pair of faucet outlets, a UV tube 24 to sterilize the outlets and bay, and mood lights 25 to illuminate liquid exiting the faucets. Dispenser 50 further includes a cradle 26 for receiving interchangeable appliance modules or cassettes 28 (FIGS. 4A-D). Exterior lights 27 are also formed on dispenser 10. Dispenser 50 differs from dispenser 10 in that a built-in mover 52 for moving the entire dispenser 50 is formed in the bottom of dispenser 50. Mover 52 is illustrated in detail in FIG. 6 and, as shown, may include a spring-loaded engaging portion or handle 54.

The particular embodiments of the invention described in this document should be considered illustrative, rather than restrictive. Modification to the described embodiments may be made without departing from the spirit of the invention as defined by the following claims. In particular, although the various entertainment, utilitarian and safety features have been described as combined into a beverage dispenser, as is apparent from the following claims the invention also encompasses the features individually or in various sub-combinations.

1. A beverage dispenser comprising:
an outlet for dispensing beverages; and
an ultraviolet light source directed at the outlet for disinfecting the outlet.

2. A beverage dispenser as claimed in claim 1, and further comprising:
a tray configured underneath the outlet for supporting containers to receive beverages dispensed from the outlet,
wherein the ultraviolet light source is directed at the tray for disinfecting the tray.

3. A beverage dispenser as claimed in claim 1, and further comprising:
automatic control means for illuminating the ultraviolet source after use of the faucet.

4. A beverage dispenser as claimed in claim 3, wherein the automatic control means illuminates the ultraviolet light source for a fixed interval after use of the faucet.

5. A beverage dispenser as claimed in claim 3, wherein the automatic control means illuminates the ultraviolet light source after a predetermined period of nonuse of the beverage dispenser.

6. A beverage dispenser comprising:
an outlet for dispensing beverages;
a tray for supporting a container underneath the outlet; and
a light source for illuminating a path of a beverage between the outlet and the tray.

7. A beverage dispenser as claimed in claim 6, wherein the light source is blue.

8. A beverage dispenser as claimed in claim 6, wherein there is a cold water outlet and a hot water outlet, and wherein there is a red light source for illuminating water exiting the hot water outlet and a blue light source for illuminating water exiting the cold water outlet.

9. A beverage dispenser as claimed in claim 6, and further comprising:
fluorescent lights mounted on the exterior of the beverage dispenser.

10. A water dispenser comprising:
a dispensing bay including faucet outlets mounted above a container support tray; and
a water bottle storage cabinet underneath the dispensing bay for storing a water bottle, the storage cabinet including a door for opening and closing the cabinet, a movable trolley for supporting and moving the water bottle, and a hinged ramp to facilitate entry and exit of the trolley into and out of the cabinet.

11. A beverage dispenser comprising:
a dispensing bay for dispensing beverages;
a cradle for securing an appliance module to the dispenser; and
an interface for communication of data or power between the appliance module and the dispenser.

12. A beverage dispenser as claimed in claim 11, wherein the appliance module is selected from a group of interchangeable appliance modules including a CD player, a soothing sounds player, an alarm clock and a radio.

13. A beverage dispenser as claimed in claim 11, and further comprising:
a locking tab for securing the appliance module in the cradle.

14. A counter-top beverage dispenser comprising:
a dispensing bay for dispensing beverages; and
a built-in mover underneath the dispensing bay to facilitate easy movement the dispenser.

15. A beverage dispenser comprising:
an outlet for dispensing beverages;
a tray for supporting a container underneath the outlet;
an ultraviolet light source directed at the outlet for disinfecting the outlet;
a light source for illuminating a path of a beverage between the outlet and the tray;
exterior lights mounted on an exterior of the dispenser;
a cradle for securing an appliance module to the dispenser; and
an interface for communication of data or power between the appliance module and the dispenser.

16. A beverage dispenser as claimed in claim 15, and further comprising:
a bottle storage cabinet underneath the dispensing bay for storing a bottle, the storage cabinet including a door for opening and closing the cabinet, a movable trolley for supporting and moving the bottle, and a hinged ramp to facilitate entry and exit of the trolley into and out of the cabinet.

17. A beverage dispenser as claimed in claim 15, and further comprising:
a built-in mover underneath the dispenser to facilitate easy movement the dispenser.
